(19) United States
(12) Patent Application Publication
(10) Pub. No.: US 2024/0156871 A1
(43) Pub. Date: May 16, 2024

(54) CYTOKINE-INDUCED KILLER CELLS

(71) Applicant: Ingo Schmidt-Wolf, Bonn (DE)

(72) Inventor: Ingo Schmidt-Wolf, Bonn (DE)

(21) Appl. No.: 18/550,832

(22) PCT Filed: Mar. 15, 2022
(86) PCT No.: PCT/EP2022/056603

§ 371 Date: Sep. 15, 2023

(30) Foreign Application Priority Data
Mar. 17, 2021 (EP) 21163132.0

(51) Int. Cl. A61K 35/17; A61K 39/00; A61P 33/06; C07K 14/725; C12N 5/0783

(52) U.S. Cl. CPC A61K 35/17; A61K 39/4613; A61K 39/464411; A61P 33/06; C07K 14/7051; C12N 5/0646

(57) ABSTRACT

The present invention relates to cytokine induced Killer cells (CIKs) for use in the treatment of diseases caused by a Plasmodium infection.

The present invention is directed to Cytokine-Induced Killer cells (CIK), for use in the treatment of diseases caused by a Plasmodium infection.

Mankind has been suffering from diseases caused by Plasmodium infections since ages and since no vaccine against diseases caused by Plasmodium infections is known so far, the provision of medication against diseases caused by Plasmodium infections is of major importance.

The major disease caused by a Plasmodium infection is malaria, which is known in the forms of malaria tropica, malaria quartana, malaria tertiana and/or malaria quotidiana.

There is a recent hypothesis of emergence of virulent Plasmodium falciparum in Africa within the past 6000 years due to a cascade of changes in human behaviour and mosquito transmission (Deirdre et al. Science. 2003 Apr. 11; 300(5617):318-21). This virulence together with transmission of malaria through population inflows from highly endemic areas, which have been poorly controlled, poses great challenges in the fight against the disease by national malaria control programs (Aayush Khadka et al. Malar J (2018) 17:224). Early 2020 WHO reports showed that the global burden of malaria as of 2018 stood at an estimate of 228 million cases and an estimated number of 405 000 deaths, with children below 5 years of age being the most vulnerable group affected by the disease. Tropical African Region carries a disproportionately high share of the global malaria burden.

In 2019, ECDC's surveillance reports showed that for 2018, 8349 malaria cases were registered in the EU/EEA, with majority of cases being imported from endemic regions. German surveillance data also showed a sharp increase of malaria cases in 2014 and 2015 due to the increased arrival of refugees from malaria endemic countries (see: https://www.ecdc.europa.eu/en/publications-data/malaria-annual-epidemiologicalreport-2018 and Vygen-Bonnet et al. Changes in malaria epidemiology in Germany, 2001-2016: a time series analysis. 2018 Jan. 15).

The unicellular protozoan parasite (Plasmodium falciparum) of humans is the deadliest species of Plasmodium that cause malaria in humans. It has been hypothesized that malarial symptoms are caused by an endogenous form of vitamin A intoxication induced by the parasites following their egress from the liver. Following this hypothesis, the parasites emerge from the liver packed with vitamin A and use retinoic acid (RA) as a cell membrane destabilizer to invade the red blood cells hence, haemolysis and anaemia. Other symptoms of the disease—e.g., fever, headache, muscle aches, gastrointestinal symptoms, seizures, coma, respiratory distress, and retinopathy—may similarly reflect parasite-induced vitamin A toxicity in the brain and other organs, following the transport of RBCs throughout the body and the release of RA(s) into the circulation (Mawson A et al. Pathogens and global health, 107(3), 122-129). P. falciparum modifies the surface of the infected RBCs and creates an adhesive phenotype, which removes the parasite from circulation for almost half of the asexual life cycle, a unique time frame among the malaria parasites (Milner D et al. Cold Spring Harbor perspectives in medicine, 8(1), a025569).

It was one objective of the present invention to provide a medicament for the treatment diseases caused by a Plasmodium infection, especially for a treatment of malaria.

This objective has been solved by Cytokine-Induced Killer (CIKs) cells that can be used in the treatment of a Plasmodium infection, especially for a Plasmodium falciparum infection, in particular for the treatment of malaria.

The ensuing description provides some embodiment(s) of the invention, and is not intended to limit the scope, applicability or configuration of the invention or inventions. Various changes may be made in the function and arrangement of elements without departing from the scope of the invention as set forth herein.

The terminology used in the description of the disclosure herein is for the purpose of describing particular embodiments only and is not intended to be limiting of the subject matter. As used in this description and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” can also be understood as “consisting of” when used in this specification.

CIK cells have been first described by our group in Schmidt-Wolf I. G. H., Negrin R. S., Kiem H., Blume K. G., Weissman I. L. (1991) Use of SCID mouse/human lymphoma model to evaluate cytokine-induced killer cells with potent antitumor cell activity. J. Exp. Med., 174(1), 139-149, the disclosure of which is herein fully incorporated by reference.

Cytokine-induced killer cells are CD3-and CD56-positive, non-major histocompatibility complex (MHC)-restricted, natural killer (NK)-like T lymphocytes, generated ex-vivo by incubation of peripheral blood lymphocytes (PBLs) with anti-CD3 monoclonal antibody, interleukin (IL)-2, IL-1, and interferon gamma (IFN-gamma) and then expanded. (see: https://www.cancer.gov/publications/dictionaries/cancer-drug/def/cytokine-inducedkiller-cells)

One potential alternative to reconstitute host immunity is adoptive immunotherapy, which can eliminate cancer cells through transfusing in vitro expanded and activated immune cells, such as CIKs. natural killers (NKs) cytotoxic lymphocytes (CTLs), and tumor-infiltrating lymphocytes (TILs) (Dan Chen et al. Cytokine-induced killer cells as a feasible adoptive immunotherapy for the treatment of lung cancer).

Plasmodium is usually understood as a genus of unicellular eukaryotes that are obligate parasites of vertebrates and insects.

Plasmodium is a member of the phylum Apicomplexa, a large group of parasitic eukaryotes. Within Apicomplexa, Plasmodium is in the order Haemosporida and family Plasmodiidae. Over 200 species of Plasmodium have been described, many of which have been subdivided into 14 subgenera based on parasite morphology and host range.

Species of Plasmodium are distributed globally wherever suitable hosts are found. Insect hosts are most frequently mosquitoes of the genera Culex and Anopheles. Vertebrate hosts include reptiles, birds, and mammals.

The life cycles of Plasmodium species involve development in a blood-feeding insect host, which then injects parasites into a vertebrate host during a blood meal. Parasites grow within a vertebrate body tissue (often the liver) before entering the bloodstream to infect red blood cells. The ensuing destruction of host red blood cells can result in diseases like malaria. During this infection, some parasites are picked up by a blood-feeding insect (mosquitoes in majority cases), continuing the life cycle.

In one embodiment, the Plasmodium infection is selected from infections with Plasmodium falciparum, Plasmodium malariae, Plasmodium ovale, Plasmodium vivax and/or Plasmodium knowlesi, in particular Plasmodium falciparum.

In one embodiment the diseases caused by a Plasmodium infection is malaria.

Malaria is usually understood as a mosquito-borne infectious disease that affects humans and other animals as the mosquito infect the patient with a Plasmodium species. Malaria causes symptoms that typically include fever, tiredness, vomiting, and headaches. In severe cases, it can cause yellow skin, seizures, coma, or death. Symptoms usually begin ten to fifteen days after being bitten by an infected mosquito. If not properly treated, people may have recurrences of the disease months later. In those who have recently survived an infection, reinfection usually causes milder symptoms. This partial resistance disappears over months to years if the person has no continuing exposure to malaria.

As mentioned above, malaria parasites belong to the genus Plasmodium (phylum Apicomplexa). In humans, malaria is usually caused by P. falciparum, P. malariae, P. ovale, P. vivax and P. knowlesi. Among those infected, P. falciparum is the most common species identified (˜75%) followed by P. vivax (˜20%).

Although P. falciparum traditionally accounts for the majority of deaths, recent evidence suggests that P. vivax malaria is associated with potentially life-threatening conditions about as often as with a diagnosis of P. falciparum infection. P. vivax proportionally is more common outside Africa.

There have been documented human infections with several species of Plasmodium from higher apes. However, except for P. knowlesi these are mostly of limited public health importance.

In one embodiment of the invention the diseases caused by a Plasmodium infection is malaria selected from Malaria tropica, Malaria quartana, Malaria tertiana and/or Malaria quotidiana.

In one embodiment, the invention is characterized in that the CIKs are used for the treatment of diseases caused by a Plasmodium infection is Malaria, in particular selected from Malaria tropica, Malaria quartana, Malaria tertiana and/or Malaria quotidiana, wherein the patient suffering therefrom is resistant against known malaria medications.

Known malaria medications in this context comprise tissue schizontocides, like primaquine, pyrimethamine, sulphonamides and/or proguanil, hypnozoitocides, like primaquine, blood schizontocides, like quinolines such as mepacrine, chloroquine, amodiaquine, mefloquine, quinine, pyronaridine and/or lumefantrine, gametocides, like primaquine, and sporontocides, like primaquine and/or pyrimethamine.

Other known malaria medications in this context comprise Chloroquin, Mefloquin Doxycyclin and/or a mixture of atovaquone/proguanil known under the name malarone.

In one embodiment of the invention, the CIK are derived from eukaryotic cells, preferably Peripheral Blood Mononuclear Cells, that have been treated with at least one of the cytokines IFNγ, anti-CD3, IL1-ß, IL-2, IL-4, GM-CSF and/or TNFa as described in “CIK cells have been first described by our group in Schmidt-Wolf I. G. H., Negrin R. S., Kiem H., Blume K. G., Weissman I. L. (1991) Use of SCID mouse/human lymphoma model to evaluate cytokine-induced killer cells with potent antitumor cell activity. J. Exp. Med., 174(1), 139-149)”.

In one embodiment of the invention, the CIKs are CD3- and/or CD56-positive, preferably CD-3 positive.

CIK cell cultures are heterogeneous populations which can be divided into CD3+CD56+, CD3-CD56+ and CD3+CD56− cells. CD3+CD56+ natural killer T (NKT) cells are the main population involved in the non-major histocompatibility complex (MHC)-restricted anti-tumor activity of CIK cultures. On the cell surface of this subset both T-cell receptor (TCR) and CD8 can be found.

Furthermore, they also express various NK-cell markers like NKG2D. Those cells have been found to be derived from CD3+CD56− T-cells. The CD3+CD56− population express typical T-cell related markers like CD4, CD8 and TCR. In a similar fashion CD3−CD56+ cells have phenotypes similar to natural killer (NK) cells, expressing common NK-cell receptors like CD16.

In one embodiment of the invention, the CIKs are CD3+CD56+.

In one embodiment of the invention, the CIKs are CD3−CD56+.

In one embodiment of the invention, the CIKs are CD3+CD56−

In one embodiment of the invention, the CIKs are CD3+CD56+ natural killer T (NKT) cells, wherein on the cell surface of this subset both T-cell receptor (TCR) and CD8 can be found.

In one embodiment of the invention, the CIKs are CD3+CD56+ natural killer T (NKT) cells, wherein on the cell surface of this subset both T-cell receptor (TCR), CD4 and CD8 can be found.

In one embodiment of the invention, the CIKs are CD3−CD56+ cells have phenotypes similar to natural killer (NK) cells, expressing common NK-cell receptors like CD16.

In one embodiment of the invention, the CIK are derived from eukaryotic cells, preferably Peripheral Blood Mononuclear Cells, that have been treated with IFNγ, anti-CD3, IL1 and/or IL-2.

In one embodiment of the invention, the CIK are derived from eukaryotic cells, preferably Peripheral Blood Mononuclear Cells, that have been treated with at least one of the cytokines IFNγ, anti-CD3, IL-1ß and/or IL-2.

In one embodiment of the invention, the CIK are derived from eukaryotic cells, preferably Peripheral Blood Mononuclear Cells, that have been treated with IFN-γ, IL-1ß, IL-2 and anti-CD3 in that order.

In one embodiment of the invention, the CIK are derived from eukaryotic cells, preferably Peripheral Blood Mononuclear Cells, that have been treated with IFN-γ on day 1, IL-1ß on day 2, IL-2 on day 2 and anti-CD3 on day 2.

In one embodiment of the invention, the CIK are derived from eukaryotic cells, preferably Peripheral Blood Mononuclear Cells, that have been treated with IFN-γ on day 1, IL-1ß on day 2, IL-2 on day 2 and anti-CD3 on day 2, wherein II-2 is further added on day 3 and continuously after every 3 days.

Preferably, CIK cells are isolated and generated from peripheral blood cells.

In one embodiment, using gradient density centrifugation peripheral blood mononuclear cells (PBMCs) are isolated from the full blood.

Remaining erythrocytes are preferably removed using osmotic lysis.

Peripheral blood lymphocytes (PBLs) are preferably separated from the other PBMCs (e.g. monocytes) by adhering the PBMCs to tissue culture (TC)-treated dishes while PBLs remain in suspension.

In order to generate CIK cells out of the isolated PBLs, they get preferably treated with specific cytokines over the course of 14-21 days.

CIK cells are preferably cultured in RPMI 1640 medium, supplemented with 10% fetal bovine serum (FBS) and 25 mM HEPES buffer.

In one embodiment, on day 0 1.000 IU/ml of human recombinant IFNγ is added to the culture.

Further, in one embodiment on day 1 50 ng/ml of anti-CD3 antibody (clone OKT3), 100 IU/ml IL-1β and 600 IU/ml of IL-2.

From this point 600 IU/ml of fresh IL-2 is preferably added every third day of culture. During this 2-3 weeks maturation period, the cell-suspension is splitted and medium is exchanged as necessary.

The common volume of a CIK-suspension is 20 ml.

In another embodiment the CIKs are derived from eukaryotic cells, preferably Peripheral Blood Mononuclear Cells, that have been treated with 500 to 1500 IU/ml, preferably about 1000 IU/ml of human recombinant IFNγ on day 0 and with 10 to 200 ng/ml, preferably 50 ng/ml of anti-CD3 antibody, 10 to 200 IU/ml, preferably about 100 IU/ml IL-1β and 300 to 900 IU/ml, preferably about 600 IU/ml of IL-2 on day 1 and wherein 300 to 900, preferably about 600 IU/ml of fresh IL-2 is preferably added every third day.

In another embodiment the CIKs are derived from eukaryotic cells, preferably Peripheral Blood Mononuclear Cells, that have been treated with 500 to 1500 IU/ml, preferably about 1000 IU/ml of human recombinant IFNγ on day 0 and with 10 to 200 ng/ml, preferably 50 ng/ml of anti-CD3 antibody, 10 to 200 IU/ml, preferably about 100 IU/ml IL-1β and 300 to 900 IU/ml, preferably about 600 IU/ml of IL-2 on day 1 and wherein 300 o 900, preferably about 600 IU/ml of fresh IL-2 is preferably added every third day, wherein the cells are matured for 2-3 weeks.

Preferably during this maturation, the cell-suspension is splitted and medium is exchanged as necessary.

For the generation of one batch of mature CIK-cells a total of 10000 to 30000 IU, preferably about 20000 IU of IFNγ, 0.1 to 2 μg, preferably about 1 μg of anti-CD3 antibody, 1000 to 3000, preferably about 2000 IU of IL-1β and around 50000 to 90000, preferably about 72000 IU of IL-2 are usually required.

The present invention is now further illustrated in the following examples. The examples should not be considered as limiting the invention in any way.

### EXAMPLES

General Reagents:

CIK Medium Preparation:
RPMI 1640: remove 67.5 ml, then add:
   12.5 mL 4-(2-hydroxyethyl)-1-piperazineethanesulfonic acid (HEPES) buffer
   1% (5 mL) of Penicillin/Streptomycin solution (P/S)
   10% (50 mL) of fetal bovine serum (FBS) (aliquot)

Other Solution Preparation:
PBS-EDTA solution (1:250): 500 ml PBS+2 ml EDTA (0.5M, 500 ml)
Erythrocyte lysis buffer: 1 ml erythrocyte lysis stock+9 ml sterile distilled water
IFN-γ: ImmunoTools (cat. 11343536, 100 μg): final concentration is 1000 U/ml. +1 ml distilled water to obtain 50 aliquots (2000 U/μL, 20 μL, 20 μL/tube)
IL-1ß: Your ImmunoTools-Team company (cat. No. 11340013, 10 μg), the final concentration is 100 U/ml. 50 aliquots (20000 U/μL, 2 μL, 10 tubes) made with 100 μL sterile distilled water, then sub-aliquoted (40 U/μL, 1000 μL, 100 μL/tube) with 1 ml distilled water when used for CIK culture.
Anti-CD3: 2 μL of the stock (1 mg/mL), no further dilution.
IL-2: ImmunoTools-Team (cat. No. 11340027, 250 μg), the final concentration is 600 U/ml, 100 aliquots (1000 U/μL, 25 μL, 24 μL/test) made with 2.5 ml distilled water.

General Methods:

1. Blood Collection: Citrated blood group A+ is provided by the Hematology unit of the University Teaching hospital Bonn. Blood is centrifuged at 2000 rpm for 5 minutes to separate serum and buffy coat from red blood cells (RBCs).

RBCs and buffy coats are further purified for Plasmodium culture and CIK cells generation, respectively.

2. CIK Cells Generation: The procedure developed by the Med. 1 laboratory of AG Schmidt-Wolf of the University hospital, Bonn is used. PBMCs are treated for at least 14 days with cytokines. Maturation of CIK cells is analyzed using flow cytometry by checking surface expression of CD3, CD56 and CD8.

In particular, CIK cells were generated as follows:
1—Pipette 25 mL of PBS-EDTA solution (1:250) to a Falcon tube.
2—Add 25 mL of Blood (1:1 ratio) and mix gently.
3—In another Falcon tube pipette 15 mL of Ficoll.
4—Take 15 mL of the blood-PBS solution, and start pipetting slowly, make sure the setting in the pipette is set on “Ex”.
5—Repeat step 4, in the end the tube should be filled up to 45 mL mark, the lower 15 mL are visibly separated from the upper 30 mL of blood.
6—Centrifuge at 1000 rpm for 30 min (this will last for a total of 1 hour)
7—Take the tube out of the centrifuge gently, Avoid shaking it!
8—With a smaller pipette (5-10 mL), suck the white layer, or the buffy coat and transfer to a new 15 mL PBS/EDTA-containing tube.
9—Fill the tube up to 50 mL with PBS/EDTA and centrifuge at 800 rpm for 7 min.
10—Discard the supernatant, and add 10 mL of erythrocyte lysis buffer. Resuspend the pellet very well, and place on ice for 10 min.
11—Repeat step (9).
12—Discard the supernatant, and Resuspend the pellet in 5 mL of CIK cell medium
13—Dilute 100 μL of the cell suspension with 900 μL of PBS or Medium.
14—Take 10 μL of the diluted suspension and add it to 90 μL of trypan blue dye.
15—Take 10 μL of the solution, and count under microscope using Neubauer Chamber.
16—Calculate the total cell count as follows:

$$\frac{\overline{ce coun \times 10 \times 10 \times 5}}{4} \times {10}^{4} = total cell count in 5 mL$$

17—Day 0: Seed 75×10⁶ cells in 40 mL of CIK media in T-175 cell culture flask.
18—Wait at least 2 hours and transfer all the medium to a new T-175 flask. Add 20 μL of IFN-γ (2000 U/μL).
19—Next day add 100 μL of IL-1ß (40 U/μL per subaliquot, 1000 μL) and 2 μL anti-CD3 (1 mg/mL), as well as 24 μL of IL-2 (1000 U/μL).
20—Continue to add IL-2 on day 3 and after every 3 days.

3. Culture of Plasmodium falciparum: This is achieved following the 3D7 technique employed by the Microbiology laboratory of the University Hospital, Bonn. In this method, the parasites are cultured by infecting purified RBC in malaria culture medium (MCM=RPMI+Gentamycin+Albumax+Hypoxanthin) at 37° C. in a gaseous atmosphere (3% Oxygen, 5% Carbondioxide and 92% Nitrogen). Plasmodium continuous culture is maintained by adding non-infected RBCs to keep parasitaemia level between 1-6%. For every second intra-erythocytic life-cycle a synchronization by single sorbitol treatment is performed. Parasitemia level is measured using Giemsa stain.

4. Giemsa Staining and Microscopy: Cultured blood smear slide is fixed in ethanol for 1 minute, stained with Giemsa stain (working solution=1 ml Giemsas Azur Eosin-Methylene blue+19 ml buffer solution) for 12-15 minutes, rinsed with distilled water and air-dried. Counting of the parasites (trophozoites and schizonts) is done with the light microscope at magnification 100× (oil immersion).

5. Plasmodium and CIK cells Co-culture: E/T ratios for the co-culture is determined after the parasitemia has been done. Cytotoxicity is investigated by measuring the remaining parasitaemia level with Giemsa staining.

Co-Culture of CIK Cells and Plasmodium falciparum (Pf) Infected Erythrocytes (RBCs).
1. After centrifuging 13 days old CIK cells and the supernatant discarded, they were diluted with 1 ml of CIK cell medium.
2. 10 μl was aliquoted and stained with 90 μl of trypan blue and counted using Neubauer counter.
3. 100 μl from (1) was then aliquoted for phenotyping using flow cytometry (see Figure.
4. The supernatant from the Pf culture plate was sucked off and thin smear slide of the RBCs was prepared and air-dried.
5. The slide was fixed with acetone for 30 seconds and stained with 1:10 dilution of Giemsa for 10 minutes, after which it was washed with running water and dried.
6. Parasite counting was done at immersion oil magnification (100×) of a light microscope and quantitation done in percentage.
7. In a 96 well culture plate, 160 μl of malaria culture medium (MCM=RPMI+gentamycin+albumax+hypoxanthin)) was filled in each of the first three wells.
8. Control wells (4 and 5) were filled with 100 μl of the MCM each.
9. Into wells 1-3, 20 μl of CIK cells was added into each, and 100 ul added to well 4.
10. 20 μl of RBCs were put into well 1, 30 ul into well 2, 10 ul into well 3 and 100 ul into well 5.
11. All mixtures were mixed by gently pipetting up and down.
12. The plate was then slantly covered, placed in a gas chamber, chamber filled with gas (3% Oxygen, 5% Carbon dioxide and 92% Nitrogen) and then incubated at 37° C. for 24 hours.
13. Thin smear slides were prepared from wells 1-3 and 5, then same procedure of (5) and (6) performed.
14. 10 μl from well 4 was again stained with trypan blue to check that the CIK cells were not lysed.

Results:

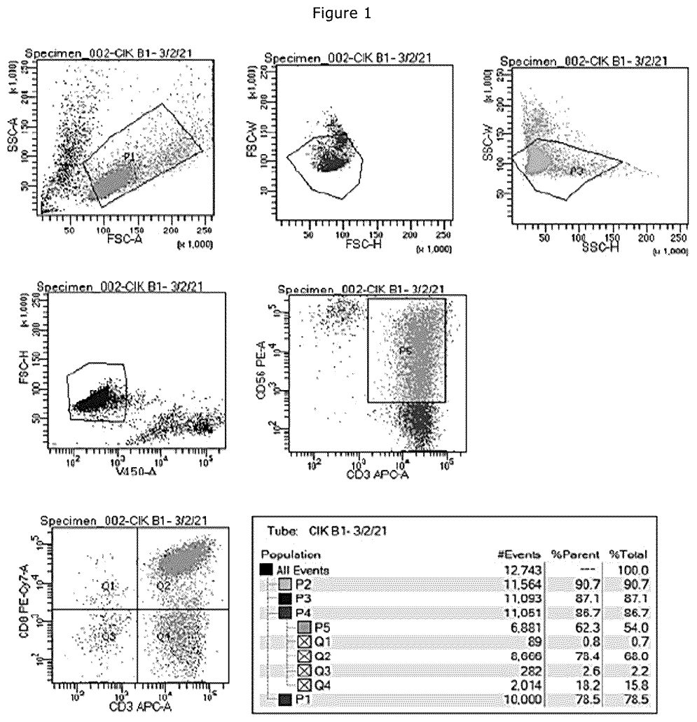

Characterization of the CIK cells is done based on the phenotypic expression of CD3 marker, as seen in FIG. 1. FIG. 1 shows a good expression of CD3 positive cells (quadrant 4) on FACS.

Number of CIK cells counted=51*10⁶ cells/ml

1 million cells ˜20 μl

Parasitaemia before co-culture=2.9% (29 parasites counted in 1000 RBCs)

Parasitaemia after co-culture as seen in table 1 below

| | Vol. of | | | Parasitemia | Parasitemia |
|---|---|---|---|---|---|
| | CIK | Vol. of | Vol. of | before co- | aftr co- |
| | cells/μl | RBCs/μl | medium/μl | culture/% | culture/% |
| Well 1 | 20.0 | 20.0 | 160.0 | 2.9 | 2.0 |
| Well 2 | 20.0 | 30.0 | 160.0 | 2.9 | 2.2 |
| Well 3 | 20.0 | 10.0 | 160.0 | 2.9 | 0.6 |
| Well 4 | 100 | — | 100 | Normal | Normal |
| (control) | | | | | |
| Well 5 | — | 100 | 100 | 2.9 | 4.0 |
| (control) | | | | | |

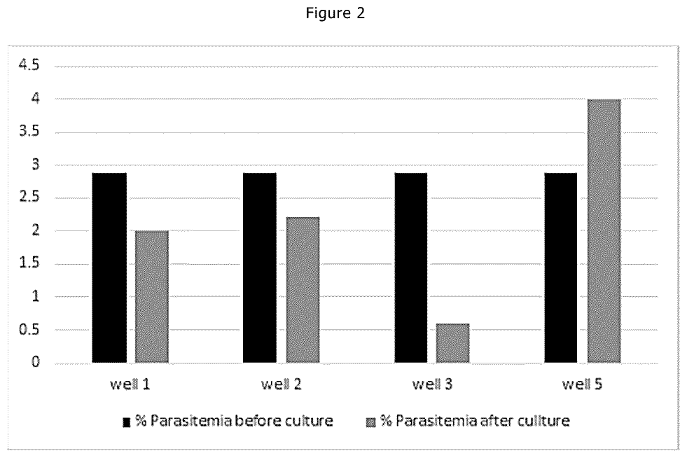

A comparison of 24 hours co-inoculation of 13 days old CIK cells and RBCs infected with Pf is given in FIG. 2.

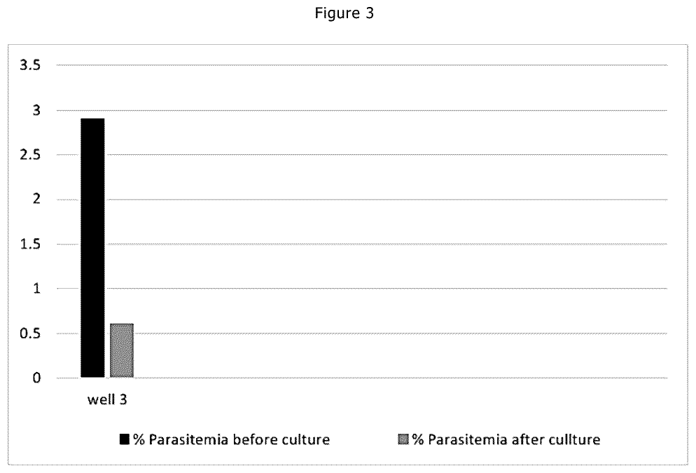

A comparison of Well 3 showing a 79.3% decrease of parasitaemia after co-culture (CIK cells to RBC ratio=2:1) is given in FIG. 3.

In a further experiment, CIK were co-cultured with Pf in RBCs for 24 hrs at CIK cells to RBCs ratios of 1:1.5, 1:1 and 1:0.5 in a 96 well culture plate.

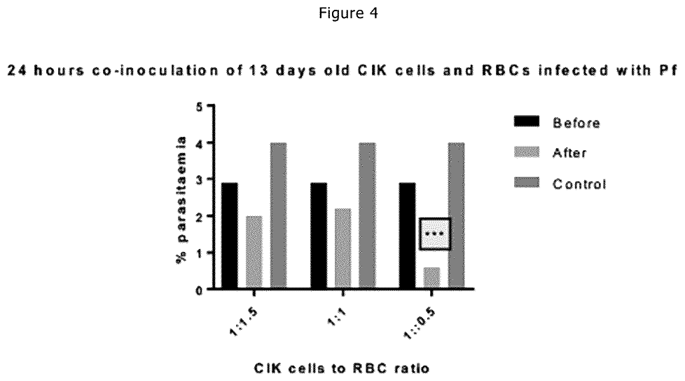

The results are given in FIG. 4.

The % parasitaemia=number of parasites counted against 1000 RBCs*100.

The marked parasitaemia decreased for 85% at a ratio of 1:0.5.

The same experiment was repeated with cells from two other donors (D1 and D2).

The marked parasitaemia decreased for 71.7% and 74.3% for D1 and d2, respectively.

The results are given in FIG. 5.

1. Use of cytokine-induced Killer (CIK) cells for the treatment of diseases caused by a Plasmodium infection in a patient in need thereof.

2. The use of claim 1, wherein the Plasmodium infection is infections with Plasmodium falciparum, Plasmodium malariae, Plasmodium ovale, Plasmodium vivax, Plasmodium knowlesi, or a combination thereof.

3. The use of claim 1, wherein the diseases caused by a Plasmodium infection is Malaria tropica, Malaria quartana, Malaria tertiana, Malaria quotidiana, or a combination thereof.

4. The use of claim 3, wherein the patient is resistant against known malaria medications.

5. The use of claim 1, wherein the CIK cells are CD3-positive, CD56-positive, or a combination thereof.

6. The use of claim 1, wherein the CIK cells are derived from eukaryotic cells.

7. The use of claim 6, wherein the eukaryotic cells are peripheral blood mononuclear cells that have been treated with IFN-γ, IL-1β, IL-2, and anti-CD3 in that order.

8. The use of claim 7, wherein the peripheral blood mononuclear cells were treated with IFN-γ on day 1, IL-1β on day 2, IL-2 on day 2, and anti-CD3 on day 2.

9. The use of claim 8, further comprising adding IL-2 on day 3 and continuously adding IL-2 after day 3 on every third day.

10. The use of claim 6, wherein the eukaryotic cells are peripheral blood mononuclear cells that have been treated with:
500 IU/ml to 1500 IU/ml human recombinant IFNγ on day 0;
10 nq/ml to 200 ng/ml anti-CD3 antibody;
10 IU/ml to 200 IU/ml IL-1β; and
300 IU/ml to 900 IU/ml IL-2 on day 1, wherein 300 IU/ml to 900 IU/ml fresh IL-2 is added every third day after day 1.

11. The use of claim 10, wherein the peripheral blood mononuclear cells are matured for 2-3 weeks.

12. The use of claim 6, wherein the eukaryotic cells are peripheral blood mononuclear cells that have been treated with IFNγ, anti-CD3, IL-1β, IL-2, or a combination thereof.

13. The use of claim 10, wherein the peripheral blood mononuclear cells have been treated with:
about 1000 IU/ml human recombinant IFNγ on day 0;
about 50 nq/ml anti-CD3 antibody;
about 100 IU/ml IL-1β; and
about 600 IU/ml IL-2 on day 1, wherein about 600 IU/ml of fresh IL-2 is added every third day after day 1.
